(19) United States
(12) Patent Application Publication
(10) Pub. No.: US 2010/0022946 A1
(43) Pub. Date: Jan. 28, 2010

(54) ENDOVASCULAR PERIAORTIC MAGNETIC GLUE DELIVERY

(76) Inventors: Ghassan S. Kassab, Zionsville, IN (US); Jose Navia, Buenos Aires (AR)

Correspondence Address: ICE MILLER LLP, ONE AMERICAN SQUARE, SUITE 3100, INDIANAPOLIS, IN, 46282-0200, US

(21) Appl. No.: 12/521,629

(22) PCT Filed: Jan. 22, 2008
(86) PCT No.: PCT/US08/00745

§ 371 Date: Jun. 29, 2009

Related U.S. Application Data
(60) Provisional application No. 60/881,474, filed on Jan. 22, 2007.

(51) Int. Cl. A61B 17/03; A61M 25/06; A61M 1/00

(52) U.S. Cl. 604/35; 606/213

(57) ABSTRACT

The disclosure of the present application includes assemblies, systems, and methods for preventing endoleak and migration. In an embodiment of an assembly for delivering a magnetic glue-like substance, the assembly comprises a catheter defining a catheter, a needle positioned within the catheter, and a needle wire, whereby a magnetic glue-like substance may be injected through a needle wire to a target site.

### PRIORITY

The present application is related to and claims the benefit of U.S. Provisional Patent Application Ser. No. 60/881,474, entitled “ENDOVASCULAR PERIAORTIC MAGNETIC GLUE DELIVERY,” filed Jan. 22, 2007.

### BACKGROUND

The disclosure of the present application relates generally to tissue support. More particularly, the disclosure of the present application relates to devices and methods for delivering a magnetic glue-like substance for an aortic tissue support device.

Aortic aneurysms are formed in a vessel when the wail of the vessel weakens, due to disease, aging, heredity, or some other process. The pressure of the blood flowing through the weakened area causes the vessel wall to balloon out, forming a blood-filled aneurysm sack. Although most aneurysms begin small, they tend to enlarge over time, and the risk of the sack rupturing increases as the aneurysms grows larger. Acute rupture of the aortic aneurysm is a life-threatening event, due to massive internal bleeding with a mortality rate of 75-80%. According to the Society of Vascular Surgeons, ruptured aneurysms account for more than 15,000 deaths in the United States each year, making the abdominal aortic aneurysm (AAA) the 13th leading cause of death in the United States. Clearly, early detection and rupture prevention are the key to the final outcome in abdominal aortic aneurysm patient. However, tile condition is under-diagnosed because most patients with AAA are asymptomatic. Consequently, the majority of the anomalies are discovered unexpectedly during routine tests or procedures. An estimated 1.7 million Americans have AAA, but only about 250,000-300,000 patients are diagnosed each year.

There is no proven medical treatment for AAA, and surgical repair has been the only common therapeutic option. A standard open repair has been associated with significant morbidity and mortality, prolonged recovery, and late complications. Because of these limitations, many patients and their physicians choose to defer operative treatment. Recently, endovascular aneurysm repair (EVAR) has become an alternative to open repair, and some studies favorably compare endovascular repair with a standard open repair. However, significant concerns exist relating to EVAR, and its value is the subject of a healthy debate.

Endovascular abdominal aortic aneurysm repair has gained acceptance as a minimally invasive alternative to open surgery in selected patients. While long-term durability remains uncertain, patients and their physicians are witling to accept a degree of uncertainly in exchange for dramatic reduction in duration of hospital stay and the need for a blood transfusion. Hence, improvements in the current EVAR devices can potentially make this approach standard for AAA repair.

Most patients diagnosed with AAA are not considered for surgery or endovascular repair unless the aneurysm is at least 5 centimeters in diameter. A 5 centimeter aneurysm is the recognized size at which the risk of rupture clearly exceeds the risk of repair. Those with a smaller aneurysm are typically followed closely with regular imaging studies. There has been much speculation over the years about the preventive use of EVAR in patients with aneurysms smaller than 5 centimeters, however, vascular surgeons have been reluctant to use EVAR for smaller aneurysms due to the concern about the long term durability of the technology and the lack of data demonstrating a clear benefit of early intervention. Moreover, although EVAR outcomes have improved over the years as physicians gain more experience with the procedure, it remains a technically demanding procedure that requires extensive training, which has limited the number of physicians qualified to perform EVAR.

Despite the shortcoming relating to training, a number of endovascular devices have been evaluated in clinical trials designed to gain approval from governmental agencies. These devices differ with respect to design features, including modularity, metallic composition, stent structure, thickness, porosity, chemical composition of the polymeric fabric, methods for attaching the fabric to the stent, and presence or absence of an active method of fixing the device to the aortic wall with bars or hooks. With consideration of the numbers of structural variations between different brands of endovascular devices, it would be remarkable if clinical outcome were not equally dissimilar. Parameters such as frequency of endoleak, long-term change in size of the aneurysm sack, reason for device migration and limb thrombosis may be linked to specific device design features. Hence, any improvements in the deployment and attachment of stent graft would increase the utility of EVAR.

Important advantages and disadvantages of EVAR are playing a big role in the decision of the treatment. Such advantages include: (1) the less-invasive nature of EVAR as compared to open repair, which translates into shorter hospitalization and recovery and lower major morbidity, (2) the general aging of the population will increase the incidence and prevalence of AAA and thoracic aortic aneurysm (TAA), (3) an increasingly-informed patient population will generate strong patient demand for minimally invasive therapy, and (4) the introduction of next-generation devices which are expected to address wider patient population (including those with thoracic disease) and reduce complications relative to current model. The disadvantages of EVAR, on the other hand, include the following: (1) the recognition that clinical literature does not support prophylactic endovascular treatment of a small aneurysm with a low risk of fracture, (2) a high rate of late complication necessitates extensive and potentially life-long post procedural follow-up (not required for open repair), and repeat intervention that makes endovascular therapy potentially more costly than open surgery, (3) current devices are not applicable to full-range of AAA patients, (4) technical demands of the approach require devices and time-consuming training that may eliminate rapid adoption of new products (particularly for specialists with a smaller case loads), and (5) the recognition that surgical conversion is complicated by the presence of the stent graft. As such, improvements in the current devices would certainly help the advantages outweigh the disadvantages.

The most important trial conducted to date is the EVAR 1 study, which randomized over 1,000 elective patients with aneurysms 5.5 centimeters in diameter or larger, comparing EVAR to open surgical repair. Thirty-day mortality published this year demonstrated a clear advantage of EVAR (1.6% vs. 4.7% for open repair). However, EVAR patients had significantly higher rates of secondary intervention (9.8% vs. 5.8%). A second version study, EVAR 2, compared EVAR with best medical treatment in patients unsuitable for surgical repair. The 12-month results for EVAR 1 are particularly noteworthy, as physicians will be looking to see if endovascular therapy is able, for the first time, to demonstrate significant survival benefit over open surgery after one year.

Despite some of its inherent drawbacks, EVAR is expected to experience robust growth over the next several years. The AAA graft market in the United States is projected to increase from $288M in 2004 to $552M in 2008. In addition, contribution from thoracic graft systems, beginning in 2007, will grow the total U.S. aortic stent market to over $670M in 2008.

A number of ongoing areas of concern with endovascular abdominal aortic repair exist, including the rate of late complications, appreciable intervention and conversion rates, and a dubious cost advantage compared to open surgery due to the need of intervention and regular patient monitoring In addition, concerns with increased device and/or procedural failure over time and that the rupture risk of one percent per year after endovascular repair is not dramatically different from the natural history of small 5 centimeter aneurysms also exist. Hence, there is high rate of secondary intervention (primarily to treat endoleaks—persistent flow within the aneurysm sack that in certain cases can lead to aneurysm rupture, if left untreated), and increasing rate of device failures over time. In addition to endoleaks, other late complications in AAA graft trials include device migration, modular component separation, graft thrombosis, bar separation, and material fatigue.

Currently, in the United States, about 60,000 AAA patients require intervention each year. The majority of the patients are treated with open surgical repair, while about 40% are treated with EVAR. Although open AAA repair is highly successful, it is also extremely invasive, with an operative mortality rate between 5-10%. Thus, patients with significant co-morbidities are generally not candidates for open repair, and as such, these patients are the primary beneficiaries of endovascular grafting or EVAR.

EVAR gained tremendous popularity in 1990 after a commercial AAA stent graft became available in the United States. After a one-year period of adjustment, however, problems with the first generation device began to surface, including migration, endoleak, and endotension. Although physicians remain confident, they have, for the most part, recovered from the disappointment associated with the first generation technology and are looking forward to future advancements in the field. Further expansion of endovascular repair is required to improve the device and good long-term results from large randomized trials comparing EVAR with open surgery. There is no doubt that a device that overcomes some of the current shortcomings of EVAR devices including as migration, endoleak, and endotension would be greatly welcomed for the treatment of aortic aneurysm.

As such, a need exists in the art for an alternative to the conventional methods of aneurysm treatment. A further need exists for a reliable, accurate, and minimally invasive device or technique of treating aneurysms that minimizes their risks of enlarging or rupturing, and effectively seals off the treated site to prevent endoleaks.

### SUMMARY

The current EVAR devices and methods are inadequate. They are prone to problems such as migration, endoleak, and endotension. In order to address this medical problem, the disclosure of the present application provides devices and methods for minimizing and/or preventing the growth or rupture of aneurysms or other vascular growth through the introduction of a magnetic glue-like substance for an aortic tissue support.

An advantage of the present approach over prior approaches is that it eliminates the need for laparoscopy, as the procedure is done percutaneously. An additional advantage is that the disclosure of the present application can not only eliminate migration and endoleak I, as referenced by prior studies, but also endoleak II by applying suction in the aneurysm sac cavity to remove blood, decrease tension, and replace the blood fluid with biologic glue in the space between the aortic aneurysm wall and the endoprosthesis. For example, and by using the disclosure of the present application, the entire body of an endograft can be made magnetic in addition to the necks.

Aneurysm size appears to be the one of the most important factors determining risk of aneurysm rupture. Changes in aneurysm dimension have been used as a surrogate marker for clinical efficacy after endovascular repair.

Endoleak is defined by the persistence of blood flow outside the lumen of the endoluminal graft but within the aneurysm sack, as determined by an imaging study. An endoleak is evidence of incomplete exclusion of the aneurysm from the circulation and may be the result of an incomplete seal between the endograft and the blood vessel wall, an inadequate connection between components of a modular prosthesis, fabric defects or porosity, or retrograde blood flow from patent aortic side branches.

Endoleaks, including their detection, potential clinical significance, and treatment remain an active area of investigation. However, although it is now evident that an endoleak may resolve spontaneously, a proportion of those that do persist have been associated with late aneurysm rupture. Endoleak I refers to inadequate seals, including those inadequate seals at the proximal and distal ends of an endograft and those at the iliac occluder plug. Endoleak II refers to flow from visceral vessels without an attachment site connection. There are also endoleaks of undefined origins where flow is visualized but the source is unidentified.

Regarding endotension, it is now appreciated as AAA may continue to enlarge after endovascular repair, even in the absence of detectable endoleak, and that this enlargement may lead to aneurysm rupture. Explanation for persistence or recurrent pressurization of an aneurysm sack includes blood flow that is below the sensitivity limits for detection with current imaging technology, or pressure transmission through thrombus, or endograft fabric. On physical examination, the aneurysm may be pulsatile and intrasac measurements may reveal pressure that approach or equal to systemic values.

Migration is defined by clinical and radiographic parameters, as a caudal movement of the proximal attachment site or cranial movement of a distal attachment site. An adhesive force with sufficient shear component would also eliminate migration.

In at least one embodiment of an assembly for delivering a magnetic glue-like substance according to the present disclosure, the assembly comprises a hollow catheter having a distal catheter aperture at a distal end of the catheter, a hollow needle positioned within the catheter, the needle having a distal needle aperture at a distal end of the needle, and at least one hollow needle wire, each needle wire having a distal needle wire aperture at a distal end of the needle wire, the at least one needle wire positioned within the needle conduit. In another embodiment, the needle is magnetic. In yet another embodiment, the needle non-magnetic. In an additional embodiment, the needle is flexible.

In at least one embodiment of an assembly for delivering a magnetic glue-like substance according to the present disclosure, the needle is slidingly engaged within the hollow catheter so that the needle may protrude from the distal catheter aperture. In another embodiment, the at least one needle wire is magnetic. In yet another embodiment, the at least one needle wire is non-magnetic. In an additional embodiment, the at least one needle wire is flexible. In a further embodiment, the at least one needle wire comprises three needle wires.

In at least one embodiment of an assembly for delivering a magnetic glue-like substance according to the present disclosure, the at least one needle wire comprises four or more needle wires. In another embodiment, the at least one needle wire is slidingly engaged within the hollow needle so that the at least one needle wire may protract from and retract into the distal needle aperture. In yet another embodiment, the needle protracts from the distal catheter aperture. In an additional embodiment, the at least one needle wire protracts from the distal needle aperture.

In at least one embodiment of an assembly for delivering a magnetic glue-like substance according to the present disclosure, the catheter comprises a hollow capsula having a distal capsula aperture at a distal end of the capsula, the capsula coupled to the catheter at the distal end of the catheter. In another embodiment, the capsula is sized and shaped to allow needle to be slidingly engaged therethrough. In yet another embodiment, the needle is slidingly engaged within the hollow capsula so that the needle may protract from and retract into the distal capsula aperture. In an additional embodiment, the at least one needle wire is slidingly engaged within the hollow capsula so that the at least one needle wire may protract from and retract into the distal capsula aperture.

In at least one embodiment of an assembly for delivering a magnetic glue-like substance according to the present disclosure, the at least one needle wire comprises an original configuration, and wherein the at least one needle wire having an original configuration is housed within the hollow capsula so that the original configuration of the at least one needle wire is altered. In another embodiment, the original configuration of the at least one needle wire may present itself after the at least one needle wire is protracted from the distal capsula aperture. In yet another embodiment, the assembly further comprises a suction/injection apparatus operably coupled to the at least one needle wire. In an additional embodiment, the suction/injection apparatus is operable to deliver a magnetic glue-like substance to the at least one needle wire. In a further embodiment, the magnetic glue-like substance may be delivered through the hollow needle wire and exit from the distal needle wire aperture of the at least one needle wire. In another embodiment, the suction/injection apparatus is operable to withdraw blood from a patient using the at least one needle wire.

In at least one embodiment of an assembly for delivering a magnetic glue-like substance according to the present disclosure, the assembly further comprises a suction/injection apparatus operably coupled to the needle. In another embodiment, the suction/injection apparatus is operable to deliver a magnetic glue-like substance to the needle. In yet another embodiment, the magnetic glue-like substance may be delivered through the hollow needle and exit from the distal needle aperture of the needle.

In at least one embodiment of an assembly for delivering a magnetic glue-like substance according to the present disclosure, the at least one needle wire is situated within the needle until a desired target site is reached upon introduction of the assembly into a patient. In another embodiment, the at least one needle wire is protracted from the needle to facilitate delivery of a magnetic glue-like substance to the target site. In yet another embodiment, the magnetic glue-like substance may be delivered through the hollow needle wire and exit from the distal needle wire aperture of the at least one needle wire. In an additional embodiment, the target site is an aneurysm sac. In a further embodiment, the at least one needle wire is protracted from the needle to facilitate the withdrawal of blood from a patient.

In at least one embodiment of an assembly for delivering a magnetic glue-like substance according to the present disclosure, the assembly comprises a hollow catheter having a distal catheter aperture at a distal end of the catheter, wherein the catheter comprises a hollow capsula having a distal capsula aperture at a distal end of the capsula, the capsula slidingly coupled to the catheter at the distal end of the catheter, a hollow needle positioned within the catheter, the needle having a distal needle aperture at a distal end of the needle, wherein the needle is slidingly engaged within the hollow catheter so that the needle may protrude from the distal catheter aperture, at least one hollow needle wire, each needle wire having a distal needle wire aperture at a distal end of the needle wire, the at least one needle wire positioned within the hollow needle, wherein the at least one needle wire is slidingly engaged within the hollow needle so that the at least one needle wire may protract from and retract into the distal needle aperture, and a suction/injection apparatus operably coupled to the at least one needle wire, the suction/injection apparatus operable to deliver a magnetic glue-like substance to the at least one needle wire.

In at least one embodiment of an system for delivering a magnetic glue-like substance according to the present disclosure, the system comprises an assembly for delivering a magnetic glue-like substance, the assembly comprising a hollow catheter having a distal catheter aperture at a distal end of the catheter, a hollow needle positioned within the catheter, the needle having a distal needle aperture at a distal end of the needle, and at least one hollow needle wire, each needle wire having a distal needle wire aperture at a distal end of the needle wire, the at least one needle wire positioned within the hollow needle, and a suction/injection apparatus operably coupled to the at least one needle wire, the suction/injection apparatus operable to deliver a magnetic glue-like substance to the at least one needle wire. In another embodiment, the needle is slidingly engaged within the hollow catheter so that the needle may protrude from the distal catheter aperture. In yet another embodiment, the at least one needle wire is slidingly engaged within the hollow needle so that the at least one needle wire may protract from and retract into the distal needle aperture. In an additional embodiment, the catheter comprises a hollow capsula having a distal capsula aperture at a distal end of the capsula, the capsula coupled to the catheter at the distal end of the catheter.

In at least one embodiment of an system for delivering a magnetic glue-like substance according to the present disclosure, the capsula is sized and shaped to allow needle to be slidingly engaged therethrough. In another embodiment, the needle is slidingly engaged within the hollow capsula so that the needle may protract from and retract into the distal capsula aperture. In yet another embodiment, the capsula is slidingly coupled to the catheter, and wherein the capsula may be pulled in a direction along the catheter opposite the distal capsula aperture to expose the at least one needle wire. In an additional embodiment, the at least one needle wire is slidingly engaged within the hollow capsula so that the at least one needle wire may protract from and retract into the distal capsula aperture.

In at least one embodiment of an system for delivering a magnetic glue-like substance according to the present disclosure, the at least one needle wire comprises an original configuration, and wherein the at least one needle wire having an original configuration is housed within the hollow capsula so that the original configuration of the at least one needle wire is altered. In another embodiment, the original configuration of the at least one needle wire may present itself after the at least one needle wire is protracted from the distal capsula aperture. In yet another embodiment, the at least one needle wire is situated within the needle until a desired target site is reached upon introduction of the assembly into a patient. In an additional embodiment, the at least one needle wire is protracted from the needle to facilitate delivery of a magnetic glue-like substance to the target site.

In at least one embodiment of an system for delivering a magnetic glue-like substance according to the present disclosure, the magnetic glue-like substance may be delivered through the at least one hollow needle wire and exit from the distal needle wire aperture of the at least one needle wire. In another embodiment, the suction/injection apparatus is further operable to withdraw blood from a patient.

In at least one embodiment of an system for delivering a magnetic glue-like substance according to the present disclosure, the system comprises an assembly for delivering a magnetic glue-like substance, the assembly comprising a hollow catheter having a distal catheter aperture at a distal end of the catheter, the catheter comprising a hollow capsula having a distal capsula aperture at a distal end of the capsula, the capsula coupled to the catheter at the distal end of the catheter, a hollow needle positioned within the catheter, the needle having a distal needle aperture at a distal end of the needle, wherein the needle is slidingly engaged within the hollow catheter so that the needle may protrude from the distal catheter aperture, and at least one hollow needle wire, each needle wire having a distal needle wire aperture at a distal end of the needle wire, the at least one needle wire positioned within the hollow needle, wherein the at least one needle wire is slidingly engaged within the hollow needle so that the at least one needle wire may protract from and retract into the distal needle aperture, and a suction/injection apparatus operably coupled to the at least one needle wire, the suction/injection apparatus operable to deliver a magnetic glue-like substance to the at least one needle wire.

In at least one embodiment of method for preventing endoleak according to the present disclosure, the method comprises the steps of introducing a catheter comprising a needle with at least one needle wire into a patient, introducing the at least one needle wire into a target site of the patient, and injecting a magnetic glue-like substance through the at least one needle wire to deliver the magnetic glue-like substance to the target site. In another embodiment, the step of introducing a catheter is performed by percutaneous arterial puncture. In yet another embodiment, the step of introducing a catheter comprises the introduction of the catheter at the landing zone of an aortic aneurism.

In at least one embodiment of method for preventing endoleak according to the present disclosure, the step of introducing at least one needle wire comprises the perforation of an aortic wall using at least one needle wire. In another embodiment, the method further comprises the step of withdrawing the at least one needle wire from the aortic wall. In an additional embodiment, the step of withdrawing the at least one needle wire externally withdraws the at least one needle wire from an adventitial surface. In another embodiment, the step of injecting a magnetic glue-like substance comprises injecting a magnetic glue-like substance to seal the perforation of the aortic wall. In an additional embodiment, the method further comprises the step of withdrawing the catheter from a patient.

In at least one embodiment of method for preventing endoleak according to the present disclosure, the method further comprises the step of withdrawing the catheter from a patient. In another embodiment, the step of injecting a magnetic glue-like substance comprises injecting a magnetic glue-like substance to seal the percutaneous arterial puncture. In another embodiment, the method further comprises the step of positioning a magnetic endograft at the target site so that the magnetic endograft is magnetically attracted to the injected magnetic glue-like substance. In yet another embodiment, the magnetic endograft is magnetically attracted to the magnetic glue-like substance outside of an aortic wall.

In at least one embodiment of method for preventing endoleak according to the present disclosure, the magnetic attraction reduces migration of the magnetic endograft from the target site. In another embodiment, the magnetic attraction eliminates migration of the magnetic endograft from the target site.

In at least one embodiment of method for preventing endoleak according to the present disclosure, the method comprises the steps of puncturing a tissue of a patient using a catheter comprising a needle with at least one needle wire, positioning the catheter at a target site of the patient, puncturing the target site using at least one needle wire, and injecting a magnetic glue-like substance through the at least one needle wire to deliver the magnetic glue-like substance to the target site.

In at least one embodiment of method for preventing endoleak according to the present disclosure, the method comprises the step of delivering a magnetic glue-like substance to the surface of a tissue, wherein the delivery is made though a catheter housing a needle with expandable needle wires capable of transporting the glue-like substance through lengths of expandable needle wires and through distal apertures of the expandable needle wires. In another embodiment, the method further comprises the step of placing a magnetic endograft around a surface of the tissue. In an additional embodiment, the magnetic endograft is placed inside an aortic lumen.

In at least one embodiment of method for preventing endoleak according to the present disclosure, the method comprises the steps of introducing a catheter comprising a needle with at least one needle wire into a patient, introducing the at least one needle wire into a target site of the patient, withdrawing blood from the target site of the patient, and injecting a magnetic glue-like substance through the at least one needle wire to deliver the magnetic glue-like substance to the target site. In another embodiment, the target site is an aneurysm sac.

As such, the disclosure of the present application provides devices and methods for minimizing and/or preventing the growth or rupture of aneurysms or other vascular growth through the introduction of a magnetic glue-like substance for an aortic tissue, minimizing or eliminating problems related endoleak, endotension, and migration.

As used throughout this application, “distal” refers to the portion of a device closest to the surface of the target tissue, while “proximal” refers to the portion of a device farthest away from the surface of the target tissue when the device is in use.

### BRIEF DESCRIPTION OF THE DRAWINGS

FIG. 1A shows an assembly for delivering a magnetic glue-like substance according to at least one embodiment of the disclosure of the present application;

FIG. 1B shows an assembly for delivering a magnetic glue-like substance with a retracted capsula according to at least one embodiment of the disclosure of the present application;

FIG. 1C shows an assembly for delivering a magnetic glue-like substance with protracted needle wires according to at least one embodiment of the disclosure of the present application;

FIG. 2 shows a front view of an aorta with an assembly for delivering a magnetic glue-like substance positioned therein according to at least one embodiment of the disclosure of the present application; and

FIG. 3 shows an assembly for delivering a magnetic glue-like substance positioned therein according to at least one embodiment of the disclosure of the present application.

### DETAILED DESCRIPTION

The disclosure of the present application provides for techniques and devices for effectively eliminating endoleak and slippage of tissue support devices. In particular embodiments and examples presented herein, such techniques and devices are described with respect to the aorta, but it must be noted that they are not limited to the aorta alone and may be applicable to any other organ or tissue that could benefit from the use of such techniques and devices.

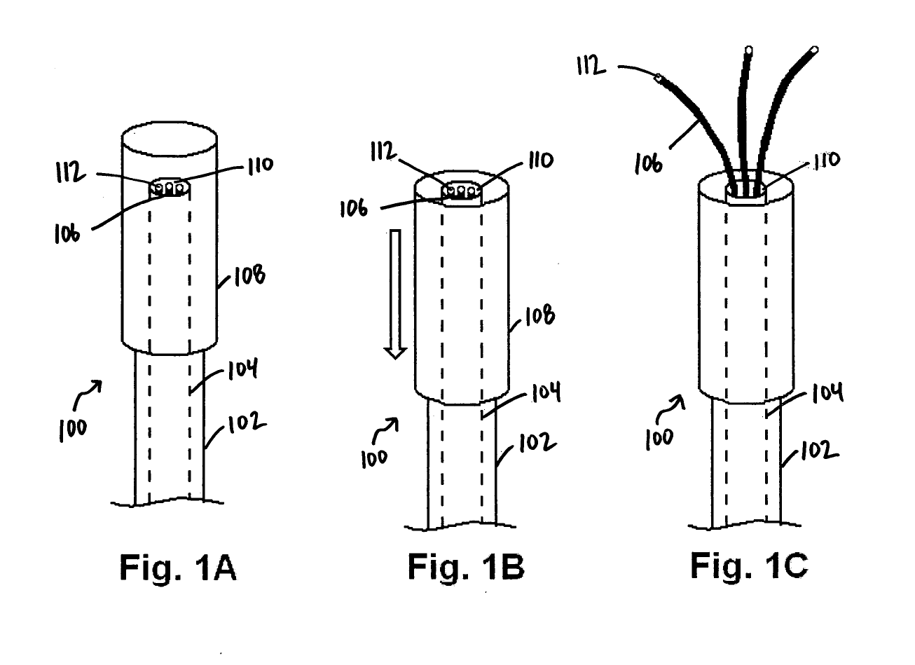

An exemplary embodiment of an assembly of the disclosure of the present application is shown in FIGS. 1A, 1B, and 1C. As shown in FIGS. 1A, 1B, and 1C, assembly 100 includes catheter 102 and needle 104. Needle 104 comprises one or more needle wires 106. Needle 104 and needle wires 106 are hollow so that a magnetic glue-like substance or other suitable substance (not shown) can pass therethrough. Needle wires 106 may be composed of any suitable material commonly used in the medical arts which would serve the functions noted herein including, but not limited to, a metallic compound.

Catheter 102 may be composed of any material known in the medical arts suitable for the application. Catheter 102 may contain an expanded capsula 108 at its distal end which may act to keep needle wires 106 in a closed, joined form. In this way, an operator can effectively manipulate catheter 102 containing needle 104 in and around a patient's body without needle wires 106 protruding and contacting surrounding tissue.

Once catheter 102 is properly positioned (e.g., within the aorta), the user may then guide needle 104 through the distal end of capsula 108 (e.g., by pulling back catheter 102 and capsula 108 in the direction shown by the arrow in FIG. 1B), thereby exposing needle wires 106 and allowing them to expand as shown in FIG. 1C.

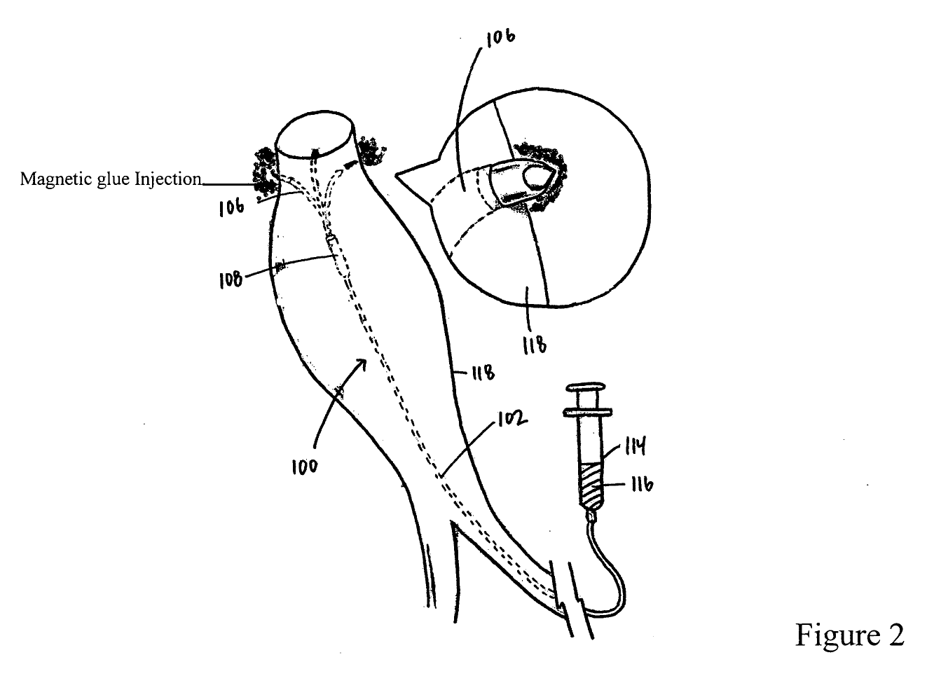

Needle 104 and needle wire 106 may possess distal needle aperture 110 and distal needle wire aperture 112, respectively, which allow suction and delivery of the magnetic glue-like substance. Needle 104 and Needle wires 106 may be non-magnetic, and may be connected to suction/injection apparatus 114 (as shown, for example, in FIG. 2) for glue delivery via the hollow interiors of needle 104 and/or needle wire 106. Capsula 108 of catheter 102 may also provide an easy displacement of needle wire 106 inside it with the diameter of capsula 108 and catheter 102 being such to allow the stable yet smooth passage of needle 104 and needle wires 106 therethrough.

Needle wires 106 may have an expanded memory as described herein (e.g., initially closed and then expanded once exposed). For example, needle wires 106 may have an original configuration (which may include, but is not limited to, a bend and/or a curve in the needle wires 106). When needle wires 106 with an original configuration are positioned within needle 104 and/or capsula 108, the original configuration may be altered (e.g, needle wires 106 may be straightened while inside needle 104 and/or capsula 108). When needle wires 106 having an original configuration are then protracted from needle 104 and/or capsula 108, the original configuration of needle wires 106 may then present themselves.

Another exemplary embodiment of an assembly of the disclosure of the present application is shown in FIG. 2. As shown in FIG. 2, assembly 100 comprises catheter 102, needle 104 (not shown), one or more needle wires 106, and capsula 108 as previously described herein. Assembly 100 may also comprise suction/injection apparatus 114 and magnetic glue-like substance 116 able to be injected by suction/injection apparatus 114. Magnetic glue-like substance 116 may exert a sufficient magnetic force so that magnetic glue-like substance 116 is positioned on the external surface of the aorta 118 (as shown in FIG. 2, including the inset), magnetic glue-like substance 116 acts to effectively stabilize the aortic surface. Magnetic glue-like substance 116 may be injected via suction/injection apparatus 114, which is connected to needle 104 (not shown) and/or needle wires 106 as shown. Magnetic glue-like substance 116 can be composed of any commonly used adhesive substance known in the medical arts which is capable of being dissolved with a magnetic compound as described. Such a magnetic compound may be chosen from any compound commonly used in the medical arts as long as it exerts the attractive properties described above. Suction/injection apparatus 114 may comprise an external syringe as shown, or it may be any other device capable of exerting pressure on a container of magnetic glue-like substance 116 thereby pushing forward the substance into needle 104 and/or expandable needle wires 106.

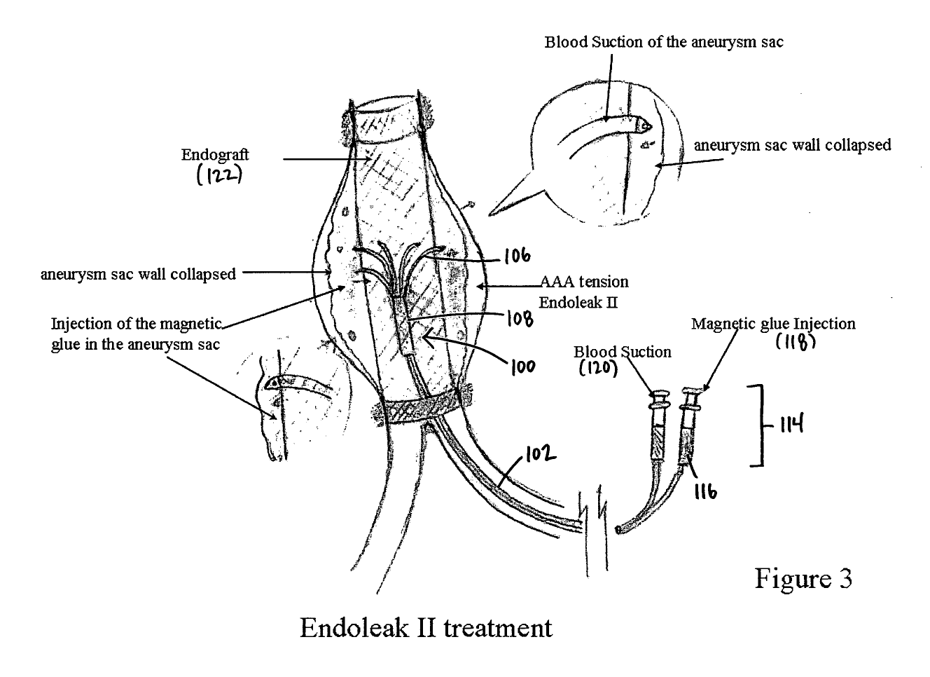

An additional exemplary embodiment of an assembly of the disclosure of the present application is shown in FIG. 3. As shown in FIG. 3, assembly 100 comprises catheter 102, needle 104 (not shown), one or more needle wires 106, and capsula 108 as previously described herein. Assembly 100 may also comprise suction/injection apparatus 114 and magnetic glue-like substance 116 able to be injected by suction/injection apparatus 114. In the embodiment shown in FIG. 3, suction/injection apparatus 114 comprises an apparatus to withdraw blood 118 from the patient (shown as “Blood Suction” on FIG. 3) and an apparatus to inject a magnetic glue-like substance 120 (shown as “Magnetic glue Injection” on FIG. 3). In this embodiment, the apparatus to withdraw blood 118 is operable to remove blood from the inside of an aneurysm sac to decrease endotension by the use of one or more needle wires 106. By decreasing the endotension within an aneurysm sac, the aneurysm sac may collapse as shown in FIG. 3.

As shown in FIG. 3, this exemplary embodiment also comprises an apparatus to inject a magnetic glue-like substance 120. Apparatus to inject a magnetic glue-like substance 120 may use one or more needle wires 106 to inject a magnetic glue-like substance 116 into an aneurysm sac. The magnetic glue-like substance 116 may replace some or all of the blood removed from by the apparatus to withdraw blood 118 from the patient. The injection of a magnetic glue-like substance 116 may seal the cavity (aneurysm sac) and adhere it to the wall of an endograft 122, effectively occluding the vessel connection responsible for endoleak II (endotension).

As discussed herein for the embodiments shown in FIGS. 2 and 3, and as can be appreciated as pertaining to other embodiments, the needle wires 106 may puncture an aneurysm sac, or another vessel or organ, from inside the lumen of the endograft wall. These punctures may then be sealed using magnetic glue-like substance 116.

The following steps are illustrative of the way in which magnetic glue-like substance 116 may be introduced to the aorta external or periaortic surface via needle 104 and apparatus as described herein: (1) percutaneous arterial puncture, (2) retrograde introduction of assembly 100 placed at the landing zone of the aortic aneurysm (i.e., health aneurysm neck), (3) deployment of needle wires 106 from within capsula 108, (4) perforation of the aortic wall with the distal ends of needle wires 106, (5) delivery of magnetic glue-like substance 116, (6) withdrawal (e.g., pulling back) of apparatus, and (7) sealing the aortic puncture with magnetic glue-like substance 116. The same or similar procedure may be repeated in the distal portion of the aortic aneurysm landing zone. In another exemplary method, the step of withdrawing blood from a patient may occur prior to the delivery of magnetic glue-like substance 116. Once magnetic glue-like substance 116 has been injected, the delivery of an endograft, a tissue support device having ferromagnetic ends, or an entire device can be performed. The endograft may be placed on the inner surface of the aortic wall. The proximal and distal ends of the endograft are not in direct contact with magnetic glue-like substance 116. On the contrary, magnetic glue-like substance 116 would be on the outside of the aortic wall and the endograft would be on the inside of the lumen in the aorta. This creates a “sandwich effect” between the magnetic glue-like substance 116 (outside of the aortic wall), the aortic wall, and the ferromagnetic portion of the endograft (inside of the aortic lumen). In this way, the endograft or tissue support device can be magnetically attracted to the magnetic glue-like substance 116 sufficiently enough to prevent slippage on or dislocation from the aortic wall.

Thus, the distal ends of needle wires 106 must be capable of puncturing the aortic wall in order to access the outer surface of the wall for delivery of magnetic glue-like substance 116. Once the target location is reached, capsula 108 may be slid down resulting in the expansion of needle wires 106. The needle 104 and/or needle wires 106 should be stiff enough so as to avoid clumsiness in targeting and puncturing the aorta. The expanded design of needle 104 and/or needle wires 106 should be engineered so as to maximize the surface area of the aorta receiving the magnetic glue-like substance 116. Also, needle 104 may also be capable of transporting magnetic glue-like substance 116 along the length of needle 104 and through the distal apertures of needle wires 106 as well.

It can be appreciated that any embodiment of an apparatus, device, assembly, suction/injection apparatus, and/or system as referenced herein may be composed of one or more suitable materials commonly used in the medical arts, including biocompatible materials as may be required for one or more individual aspects of the apparatus, device, assembly, and/or system.

It can also be appreciated that any embodiment of an apparatus, device, assembly, suction/injection apparatus, system, and/or method as described herein may be suitable for other organs and/or vessels not previously described herein.

The foregoing disclosure of the exemplary embodiments of the present application has been presented for purposes of illustration and description and can be further modified within the scope and spirit of this disclosure. It is not intended to be exhaustive or to limit the present disclosure to the precise forms disclosed. This application is therefore intended to cover any variations, uses, or adaptations of a device, system and method of the present application using its general principles. Further, this application is intended to cover such departures from the present disclosure as may come within known or customary practice in the art to which this system of the present application pertains. Many variations and modifications of the embodiments described herein will be apparent to one of ordinary skill in the art in light of the above disclosure. The scope of the present disclosure is to be defined only by the claims appended hereto, and by their equivalents.

Further, in describing representative embodiments of the present disclosure, the specification may have presented the method and/or process of the present disclosure as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present disclosure should not be limited to the performance of their steps in the order written, and one skilled in the art can readily appreciate that the sequences may be varied and still remain within the spirit and scope of the present disclosure.

What is claimed is:

1. An assembly for delivering a magnetic glue-like substance, the assembly comprising:
a hollow catheter having a distal catheter aperture at a distal end of the catheter;
a hollow needle positioned within the catheter, the needle having a distal needle aperture at a distal end of the needle; and
at least one hollow needle wire, each needle wire having a distal needle wire aperture at a distal end of the needle wire, the at least one needle wire positioned within the needle conduit.

2. The assembly of claim 1, wherein the needle is magnetic.

3. The assembly of claim 1, wherein the needle non-magnetic.

4. The assembly of claim 1, wherein the needle is flexible.

5. The assembly of claim 1, wherein the needle is slidingly engaged within the hollow catheter so that the needle may protrude from the distal catheter aperture.

6. The assembly of claim 1, wherein the at least one needle wire is magnetic.

7. The assembly of claim 1, wherein the at least one needle wire is non-magnetic.

8. The assembly of claim 1, wherein the at least one needle wire is flexible.

9. The assembly of claim 1, wherein the at least one needle wire comprises three needle wires.

10. The assembly of claim 1, wherein the at least one needle wire comprises four or more needle wires.

11. The assembly of claim 1, wherein the at least one needle wire is slidingly engaged within the hollow needle so that the at least one needle wire may protract from and retract into the distal needle aperture.

12. The assembly of claim 1, wherein the needle protracts from the distal catheter aperture.

13. The assembly of claim 12, wherein the at least one needle wire protracts from the distal needle aperture.

14. The assembly of claim 1, wherein the catheter comprises a hollow capsula having a distal capsula aperture at a distal end of the capsula, the capsula coupled to the catheter at the distal end of the catheter.

15. The assembly of claim 14, wherein the capsula is sized and shaped to allow needle to be slidingly engaged therethrough.

16. The assembly of claim 14, wherein the needle is slidingly engaged within the hollow capsula so that the needle may protract from and retract into the distal capsula aperture.

17. The assembly of claim 14, wherein the at least one needle wire is slidingly engaged within the hollow capsula so that the at least one needle wire may protract from and retract into the distal capsula aperture.

18. The assembly of claim 17, wherein the at least one needle wire comprises an original configuration, and wherein the at least one needle wire having an original configuration is housed within the hollow capsula so that the original configuration of the at least one needle wire is altered.

19. The assembly of claim 18, wherein the original configuration of the at least one needle wire may present itself after the at least one needle wire is protracted from the distal capsula aperture.

20. The assembly of claim 1, further comprising a suction/injection apparatus operably coupled to the at least one needle wire.

21. The assembly of claim 20, wherein the suction/injection apparatus is operable to deliver a magnetic glue-like substance to the at least one needle wire.

22. The assembly of claim 20, wherein the magnetic glue-like substance may be delivered through the hollow needle wire and exit from the distal needle wire aperture of the at least one needle wire.

23. The assembly of claim 20, wherein the suction/injection apparatus is operable to withdraw blood from a patient using the at least one needle wire.

24. The assembly of claim 1, further comprising a suction/injection apparatus operably coupled to the needle.

25. The assembly of claim 24, wherein the suction/injection apparatus is operable to deliver a magnetic glue-like substance to the needle.

26. The assembly of claim 24 wherein the magnetic glue-like substance may be delivered through the hollow needle and exit from the distal needle aperture of the needle.

27. The assembly of claim 1, wherein the at least one needle wire is situated within the needle until a desired target site is reached upon introduction of the assembly into a patient.

28. The assembly of claim 27, wherein the at least one needle wire is protracted from the needle to facilitate delivery of a magnetic glue-like substance to the target site.

29. The assembly of claim 28, wherein the magnetic glue-like substance may be delivered through the hollow needle wire and exit from the distal needle wire aperture of the at least one needle wire.

30. The assembly of claim 27, wherein the target site is an aneurysm sac.

31. The assembly of claim 27, wherein the at least one needle wire is protracted from the needle to facilitate the withdrawal of blood from a patient.

32. An assembly for delivering a magnetic glue-like substance, the assembly comprising:
a hollow catheter having a distal catheter aperture at a distal end of the catheter, wherein the catheter comprises a hollow capsula having a distal capsula aperture at a distal end of the capsula, the capsula slidingly coupled to the catheter at the distal end of the catheter;
a hollow needle positioned within the catheter, the needle having a distal needle aperture at a distal end of the needle, wherein the needle is slidingly engaged within the hollow catheter so that the needle may protrude from the distal catheter aperture;
at least one hollow needle wire, each needle wire having a distal needle wire aperture at a distal end of the needle wire, the at least one needle wire positioned within the hollow needle, wherein the at least one needle wire is slidingly engaged within the hollow needle so that the at least one needle wire may protract from and retract into the distal needle aperture; and
a suction/injection apparatus operably coupled to the at least one needle wire, the suction/injection apparatus operable to deliver a magnetic glue-like substance to the at least one needle wire.

33. A system for delivering a magnetic glue-like substance, the system comprising:
an assembly for delivering a magnetic glue-like substance, the assembly comprising:
a hollow catheter having a distal catheter aperture at a distal end of the catheter;
a hollow needle positioned within the catheter, the needle having a distal needle aperture at a distal end of the needle; and
at least one hollow needle wire, each needle wire having a distal needle wire aperture at a distal end of the needle wire, the at least one needle wire positioned within the hollow needle; and
a suction/injection apparatus operably coupled to the at least one needle wire, the suction/injection apparatus operable to deliver a magnetic glue-like substance to the at least one needle wire.

34. The system of claim 33, wherein the needle is slidingly engaged within the hollow catheter so that the needle may protrude from the distal catheter aperture.

35. The system of claim 33, wherein the at least one needle wire is slidingly engaged within the hollow needle so that the at least one needle wire may protract from and retract into the distal needle aperture.

36. The system of claim 33, wherein the catheter comprises a hollow capsula having a distal capsula aperture at a distal end of the capsula, the capsula coupled to the catheter at the distal end of the catheter.

37. The system of claim 36, wherein the capsula is sized and shaped to allow needle to be slidingly engaged therethrough.

38. The system of claim 36, wherein the needle is slidingly engaged within the hollow capsula so that the needle may protract from and retract into the distal capsula aperture.

39. The system of claim 36, wherein the capsula is slidingly coupled to the catheter, and wherein the capsula may be pulled in a direction along the catheter opposite the distal capsula aperture to expose the at least one needle wire.

40. The system of claim 36, wherein the at least one needle wire is slidingly engaged within the hollow capsula so that the at least one needle wire may protract from and retract into the distal capsula aperture.

41. The system of claim 40, wherein the at least one needle wire comprises an original configuration, and wherein the at least one needle wire having an original configuration is housed within the hollow capsula so that the original configuration of the at least one needle wire is altered.

42. The system of claim 41, wherein the original configuration of the at least one needle wire may present itself after the at least one needle wire is protracted from the distal capsula aperture.

43. The system of claim 33, wherein the at least one needle wire is situated within the needle until a desired target site is reached upon introduction of the assembly into a patient.

44. The system of claim 43, wherein the at least one needle wire is protracted from the needle to facilitate delivery of a magnetic glue-like substance to the target site.

45. The system of claim 33, wherein the magnetic glue-like substance may be delivered through the at least one hollow needle wire and exit from the distal needle wire aperture of the at least one needle wire.

46. The system of claim 33, wherein the suction/injection apparatus is further operable to withdraw blood from a patient.

47. A system for delivering a magnetic glue-like substance, the system comprising:
an assembly for delivering a magnetic glue-like substance, the assembly comprising:
a hollow catheter having a distal catheter aperture at a distal end of the catheter, the catheter comprising a hollow capsula having a distal capsula aperture at a distal end of the capsula, the capsula coupled to the catheter at the distal end of the catheter;
a hollow needle positioned within the catheter, the needle having a distal needle aperture at a distal end of the needle, wherein the needle is slidingly engaged within the hollow catheter so that the needle may protrude from the distal catheter aperture; and
at least one hollow needle wire, each needle wire having a distal needle wire aperture at a distal end of the needle wire, the at least one needle wire positioned within the hollow needle, wherein the at least one needle wire is slidingly engaged within the hollow needle so that the at least one needle wire may protract from and retract into the distal needle aperture; and
a suction/injection apparatus operably coupled to the at least one needle wire, the suction/injection apparatus operable to deliver a magnetic glue-like substance to the at least one needle wire.

48. A method for preventing endoleak, the method comprising the steps of:
introducing a catheter comprising a needle with at least one needle wire into a patient;
introducing the at least one needle wire into a target site of the patient; and
injecting a magnetic glue-like substance through the at least one needle wire to deliver the magnetic glue-like substance to the target site.

49. The method of claim 48, wherein the step of introducing a catheter is performed by percutaneous arterial puncture.

50. The method of claim of claim 48, wherein the step of introducing a catheter comprises the introduction of the catheter at the landing zone of an aortic aneurism.

51. The method of claim 48, wherein the step of introducing at least one needle wire comprises the perforation of an aortic wall using at least one needle wire.

52. The method of claim 51, further comprising the step of withdrawing the at least one needle wire from the aortic wall.

53. The method of claim 52, wherein the step of withdrawing the at least one needle wire externally withdraws the at least one needle wire from an adventitial surface.

54. The method of claim 52, wherein the step of injecting a magnetic glue-like substance comprises injecting a magnetic glue-like substance to seal the perforation of the aortic wall.

55. The method of claim 48, further comprising the step of withdrawing the catheter from a patient.

56. The method of claim 49, further comprising the step of withdrawing the catheter from a patient.

57. The method of claim 56, wherein the step of injecting a magnetic glue-like substance comprises injecting a magnetic glue-like substance to seal the percutaneous arterial puncture.

58. The method of claim 48, further comprising the step of positioning a magnetic endograft at the target site so that the magnetic endograft is magnetically attracted to the injected magnetic glue-like substance.

59. The method of claim 58, wherein the magnetic endograft is magnetically attracted to the magnetic glue-like substance outside of an aortic wall.

60. The method of claim 58, wherein the magnetic attraction reduces migration of the magnetic endograft from the target site.

61. The method of claim 58, wherein the magnetic attraction eliminates migration of the magnetic endograft from the target site.

62. A method for preventing endoleak, the method comprising the steps of:
puncturing a tissue of a patient using a catheter comprising a needle with at least one needle wire;
positioning the catheter at a target site of the patient;
puncturing the target site using at least one needle wire; and
injecting a magnetic glue-like substance through the at least one needle wire to deliver the magnetic glue-like substance to the target site.

63. A method for preventing endoleak and migration, the method comprising the step of delivering a magnetic glue-like substance to the surface of a tissue, wherein the delivery is made though a catheter housing a needle with expandable needle wires capable of transporting the glue-like substance through lengths of expandable needle wires and through distal apertures of the expandable needle wires.

64. The method of claim 63, further comprising the step of placing a magnetic endograft around a surface of the tissue.

65. The method of claim 64, wherein the magnetic endograft is placed inside an aortic lumen.

66. A method for preventing endoleak, the method comprising the steps of:
introducing a catheter comprising a needle with at least one needle wire into a patient;
introducing the at least one needle wire into a target site of the patient;
withdrawing blood from the target site of the patient; and
injecting a magnetic glue-like substance through the at least one needle wire to deliver the magnetic glue-like substance to the target site.

67. The method of claim 66, wherein the target site is an aneurysm sac.
